(19) United States
(12) Patent Application Publication
(10) Pub. No.: US 2007/0224252 A1
(43) Pub. Date: Sep. 27, 2007

(54) Microprojections with capillary control features and method

(76) Inventors: Joseph C. Trautman, Sunnyvale, CA (US); Richard Wilhem Janse Van Rensburg, Cambridgeshire (GB)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/391,609

(22) Filed: Mar. 27, 2006

(51) Int. Cl. A61K 9/70; A61M 31/00

(52) U.S. Cl. 424/449; 604/500

(57) ABSTRACT

The present invention provides methods and devices for reducing the coating variability of a transdermal microprojection delivery device. The device includes one or more stratum corneum-piercing microprojections, wherein each microprojection has a capillary control feature that restricts migration of a coating formulation.

### FIELD OF THE PRESENT INVENTION

The present invention relates to devices and methods for delivering a biologically active agent transdermally using a coated microprojection array. More particularly, the invention relates to devices and methods for reducing the variability in the amount of active agent coated on the microprojections, thus improving the consistency of delivered amount.

### BACKGROUND OF THE INVENTION

Active agents (or drugs) are most conventionally administered either orally or by injection. Unfortunately, many active agents are completely ineffective or have radically reduced efficacy when orally administered, since they either are not absorbed or are adversely affected before entering the bloodstream and thus do not possess the desired activity. On the other hand, the direct injection of the agent into the bloodstream, while assuring no modification of the agent during administration, is a difficult, inconvenient, painful and uncomfortable procedure which sometimes results in poor patient compliance.

As an alternative, transdermal delivery provides for a method of administering biologically active agents that would otherwise need to be delivered via hypodermic injection, intravenous infusion or orally. Transdermal delivery, when compared to oral delivery avoids the harsh environment of the digestive tract, bypasses gastrointestinal drug metabolism, reduces first-pass effects, and avoids the possible deactivation by digestive and liver enzymes.

As is well known in the art, the word “transdermal” is used that is used to refer to delivery of an active agent (e.g., a nucleic acid or other therapeutic agent such as a drug) through the skin to the local tissue or systemic circulatory system without substantial cutting or piercing of the skin, such as cutting with a surgical knife or piercing the skin with a hypodermic needle.

Transdermal agent delivery includes delivery via passive diffusion as well as by external energy sources, including electricity (e.g., iontophoresis) and ultrasound (e.g., phonophoresis). While most agents will diffuse across both the stratum corneum and the epidermis, the rate of diffusion through the stratum corneum is often the limiting step. Many compounds, in order to achieve a therapeutic dose, require higher delivery rates than can be achieved by simple passive transdermal diffusion.

One common method of increasing the passive transdermal diffusions agent flux involves pre-treating the skin with, or co-delivering with the agent, a skin permeation enhancer. A permeation enhancer, when applied to a body surface through which the agent is delivered, enhances the flux of the agent therethrough. However, the efficacy of these methods in enhancing transdermal agent flux has been limited, particularly for larger molecules.

There also have been many techniques and systems developed to mechanically penetrate or disrupt the outermost skin layers thereby creating pathways into the skin in order to enhance the amount of agent being transdermally delivered. Illustrative are skin scarification devices, or scarifiers, which typically provide a plurality of tines or needles that are applied to the skin to scratch or make small cuts in the area of application. The agent, such as a vaccine, is applied either topically on the skin, such as disclosed in U.S. Pat. No. 5,487,726, or as a wetted liquid applied to the scarifier tines, such as disclosed in U.S. Pat. Nos. 4,453,926, 4,109,655, and 3,136,314.

Other devices that use tiny skin piercing elements or microprojections to enhance transdermal agent delivery are disclosed in European Patent EP 0407063A1, U.S. Pat. No. 5,879,326 issued to Godshall, et al., U.S. Pat. No. 3,814,097 issued to Ganderton, et al., U.S. Pat. No. 5,279,544 issued to Gross, et al., U.S. Pat. No. 5,250,023 issued to Lee, et al., U.S. Pat. No. 3,964,482 issued to Gerstel, et al., Reissue 25,637 issued to Kravitz, et al., and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated by reference in their entirety.

The piercing elements disclosed in the noted references generally extend perpendicularly from a thin, flat member, such as a pad or sheet. The piercing elements are typically extremely small, some having dimensions (i.e., a microblade length and width) of only about 25-400 μm and a microblade thickness of only about 5-50 μm.

The disclosed systems generally include a reservoir for holding the active agent and a delivery system to transfer the active agent from the reservoir through the stratum corneum, such as by hollow tines or needles.

Alternatively, a formulation containing the active agent can be coated on the microprojections. Illustrative are the systems disclosed in U.S. Patent Pub. Nos. 2002/0132054, 2002/0193729, 2002/0177839, 2002/0128599, and application Ser. No. 10/045,842, which are fully incorporated by reference herein. Coated microprojection systems eliminate the necessity of a separate physical reservoir and the development of an agent formulation or composition specifically for the reservoir.

However, one challenge associated with this method of delivery lies in achieving a reproducible dose of the coated agent. Specifically, conventional means of coating can result in a significant variation in the amount of active agent loaded onto the delivery device.

For example, dip-coating is a method of applying an active agent to the microprojections of a delivery device that generally involves placing the tips of the microprojections in a reservoir of fluid. Capillary action causes the fluid to wick up the sides of the microprojections to variable heights, creating inconsistency in the amount of agent coated and the location of the agent on the microprojection array.

As will be appreciated by one having ordinary skill in the art, the distance the fluid rises up the microprojection is a function the depth the tip is dipped into the fluid, the viscosity of the fluid, the contact angle of the fluid with the microprojection material and the duration the tip is dipped into the fluid. Furthermore, the proximity of the microprojections in the array to each other creates an environment in which the fluid wicks higher in the center of the array than on the perimeter of the array.

Due to the noted effects, there can be substantial variability in the amount of active agent loaded on the microprojection delivery device.

Accordingly, it is an object of this invention to provide methods and compositions for enhancing transdermal delivery of biologically active agents using microprojection devices.

It is a further object of the invention to provide a device and method that reduces the variability in the amount of active agent coated on the microprojections.

It is another object of the invention to a device for and method of delivering a more consistent amount of a biologically active agent using a coated microprojection device.

It is yet another objection of the invention to provide a device and method that limits the capillary action when applying an active agent formulation to a microprojection delivery device.

Another object of the invention is to provide a device and method for more precisely controlling the coating depth on a microprojection.

### SUMMARY OF THE INVENTION

In accordance with the above objects and those that will be mentioned and will become apparent below, one aspect of the invention comprises a transdermal delivery device comprising a microprojection member having at least one stratum corneum-piercing microprojection with a capillary control feature, wherein the microprojection has a length running from a distal tip to a proximal end and a thickness, wherein the capillary control feature is located between the distal tip and the proximal end, and wherein the microprojection has a first width at the capillary control feature location. Preferably, the capillary control feature is located in the range of approximately 25 μm to 200 μm from the distal tip of the microprojection.

In one embodiment of the invention, the capillary control feature comprises a scribe line running perpendicular to the microprojection length. Preferably, the scribe line extends at least 50% of the first width on each side of the microprojection. The scribe line can be configured as a ridge or a trough. Also preferably, the scribe line has a thickness in the range of approximately 5 μm and 25% of the thickness of the microprojection.

In another embodiment of the invention, the capillary control feature comprises a void. Preferably, the void has a horizontal dimension up to approximately half the width of the microprojection at the location of the capillary control feature.

In yet another embodiment of the invention, the capillary control feature comprises a transition from a maximum width to a minimum width at the capillary control feature location. Preferably, the microprojection has a minimum width in the range of approximately 25% to 100% of the maximum width, and more preferably, in the range of approximately 35% to 70% of the maximum width. Even more preferably, the minimum width is approximately 50% of the maximum width. Alternately, the microprojection has a minimum width that is in the range of approximately 10 μm to 120 μm less than said maximum width.

In yet another embodiment of the invention, the capillary control feature comprises a hydrophobic coating. Presently preferred hydrophobic coatings are selected from the group consisting of polytetrafluoroethylene, parylene and silicon.

Preferably, the delivery devices of the invention further comprise a coating of a biologically active agent applied to the microprojection from the distal tip to the capillary control feature.

In another aspect of the invention, the coating is applied to the microprojection with a static contact angle greater than 20 degrees, and more preferably, between 30 and 60 degrees.

In one embodiment of the invention, the coating comprises a formulation having a biologically active agent selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8]arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

In another embodiment of the invention, the biologically active agent comprises an immunologically active agent selected from the group consisting of proteins, polysaccharide conjugates, oligosaccharides, lipoproteins, subunit vaccines, Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, Lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

The invention also comprises methods of applying a coating of a biologically active agent to a transdermal delivery device, generally including the steps of providing a microprojection member having at least one stratum corneum-piercing microprojection with a capillary control feature, wherein the microprojection has a length running from a distal tip to a proximal end, a thickness, wherein the capillary control feature is located between the distal tip and the proximal end, and wherein the microprojection has a first width at the capillary control feature location; applying a formulation of the biologically active agent to a location proximal the distal tip of the microprojection so that the formulation migrates to the capillary control feature; and drying the formulation to form a coating. Preferably, the step of applying the formulation comprises dip coating.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a microprojection member having a coating deposited on the microprojections, according to the invention;

FIG. 2 is a detail view of an embodiment of a microprojection having a scribed capillary control feature, according to the invention;

FIG. 3 is a detail view of an alternate embodiment of a microprojection having a capillary control feature comprising a void, according to the invention;

FIG. 4 is a detail view of another embodiment of a microprojection having a capillary control feature comprising a reduced width configuration, according to the invention;

FIG. 5 is a detail view of yet another embodiment of a microprojection having a capillary control feature comprising a hydrophobic coating, according to the invention;

FIGS. 6 and 7 are graphical illustrations comparing capillary rise heights for microprojections having features of the invention to prior art microprojections; and

FIGS. 8 and 9 are graphical illustrations comparing meniscus volumes for microprojections having features of the invention to prior art microprojections.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “an active agent” includes two or more such agents; reference to “a microprojection” includes two or more such microprojections and the like.

### Definitions

The term “transdermal”, as used herein, means the delivery of an agent into and/or through the skin for local or systemic therapy.

The term “biologically active agent”, as used herein, refers to a composition of matter or mixture containing an active agent or drug, which is pharmacologically effective when administered in a therapeutically effective amount.

It is to be understood that more than one biologically active agent can be incorporated into the agent source and/or coatings of this invention, and that the use of the term “active agent” in no way excludes the use of two or more such active agents or drugs.

As used herein, the term “microprojection array,” “microprojection member,” and the like, all refer to a device for delivering an active agent into or through the skin that comprises a plurality of microprojections on which the active agent can be coated. The term “microprojections” refers to piercing elements that are adapted to pierce or cut through the stratum corneum into the underlying epidermis layer, or epidermis and dermis layers, of the skin of a living animal, particularly a human.

Typically the microprojections have a blade length of less than 1000 μm, and preferably less than 500 μm. In one embodiment, the microprojections have a length in the range of 50-145 μm. The microprojections typically have a width in the range of about 75-500 μm and a thickness in the range of about 5-50 μm.

The microprojections can be formed in different shapes, for example by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s).

Exemplary methods of forming metal microprojection are disclosed in Trautman et al., U.S. Pat. No. 6,083,196; Zuck, U.S. Pat. No. 6,050,988; and Daddona et al., U.S. Pat. No. 6,091,975; the disclosures of which are incorporated by reference herein in their entirety.

Other microprojection members that can be used with the present invention are formed by etching silicon using silicon chip etching techniques or by molding plastic using etched micro-molds. Silicon and plastic microprojection members are disclosed in Godshall et al., U.S. Pat. No. 5,879,326; the disclosure of which is incorporated by reference herein.

As used herein, the terms “deliver,” “delivering,” and all variations thereof, refer to and include any means by which an active agent can be administered into or through the skin.

As used herein, the term “thickness,” as it relates to coatings, refers to the average thickness of a coating as measured over substantially all of the portion of a substrate that is covered with the coating.

Referring now to FIG. 1, there is shown one embodiment of a stratum corneum-piercing microprojection member 10 for use with the present invention. As illustrated in FIG. 1, the member 10 includes a plurality of microprojections 12 having a coating 14 disposed thereon. Coating 14 comprises a dried formulation having one or more biologically active agents. In the illustrated embodiment, the microprojections 12 extend at substantially a 90° angle from a substrate, such as sheet 16, having openings 18.

The microprojections 12 are preferably formed by etching or punching a plurality of microprojections 12 from a thin metal sheet 16 and bending the microprojections 12 out of a plane of the sheet. Metals such as stainless steel, titanium and nickel titanium alloys are preferred.

According to the invention, the coating 14 preferably covers the microprojection 12 from a capillary control feature 20 to the distal tip 22. According to the invention, the coating 14 can be formed upon the microprojections 12 by a variety of known methods. Generally, a liquid formulation is applied to microprojection 12 and then dried to form coating 14. Preferably, capillary control feature 20 is positioned within the nominal rise height of the coating formulation at a location selected to result in a desired coating depth. Thus, the applied fluid formulation wicks along the microprojection 12 until capillary control feature 20 restricts the migration.

A presently preferred means of applying a formulation to the microprojections of the invention means is dip-coating. This method generally involves immersing microprojections 12 into a coating formulation. Depending upon the properties of the coating formulation and the desired loading amount, the microprojections can be lowered into the formulation to any depth up to the capillary control feature 20. In some embodiments, it may be desirable to dip only a distal portion of the microprojection tip into the formulation.

The capillary control features of the invention are applicable to other means of applying coatings, so long as the applied formulation is fluid or otherwise susceptible to migration. As will be appreciated by one having ordinary skill in the art, the use of the capillary control features of the invention minimizes such migration and restricts the coating depth.

One alternative coating method is roller coating, which employs a roller coating mechanism that similarly limits the coating 14 to the tips of the microprojections 12. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 12 during skin piercing.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 10 and then dried.

Pattern coating can also be employed to coat the microprojections 12. The pattern coating can be applied using a dispensing system for positioning the deposited liquid onto the microprojection surface. The quantity of the deposited liquid is preferably in the range of 0.1 to 20 nl/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

The invention is directed to microprojection designs and methods having reduced coating variability. To achieve minimal coating variability, the microprojection has a capillary control feature, located so that capillary action is disrupted or minimized at the desired coating depth.

In a first embodiment, shown in FIG. 2, the invention includes a microprojection 30 having a capillary control feature comprising a scribe line 32. A scribe line 32 generally is a trough or ridge that runs substantially perpendicular to the length of the microprojection. Preferably, scribe line 32 runs continuously from edge to edge on both sides of the microprojection. Alternatively, scribe line 32 can run intermittently across at least half the distance. The thickness of scribe line 32 refers to depth of the trough or height of the ridge, and is measured as the differential from the plane of the microprojection. Preferably, the thickness of scribe line 32 is approximately equal to its width. More preferably, the thickness is in the range of approximately 5 μm and 25% of the thickness of the microprojection.

To maximize effectiveness in controlling capillary action, the edges of the scribe line preferably have a sharp configuration. Scribe line 32 is located the distance from the tip 34 of the microprojection that the fluid is intended to coat. Preferably, scribe line 32 is located in the range of approximately 25 μm to 200 μm from the distal tip 34 of the microprojection 30.

In an alternate embodiment of the invention, shown in FIG. 3, microprojection 40 has a capillary control feature comprising at least one void 42. In embodiments with a single hole, the width of the microprojection on each side of void 42 is preferably in the range of approximately 25 μm and half the width of the microprojection. Also preferably, void 42 is located so that the distal portion of the void (that closest to the tip of the microprojection) corresponds to the desired coating depth. For example, the distal portion of void 42 is preferably located in the range of approximately 25 μm to 200 μm from the tip 44 of microprojection 40.

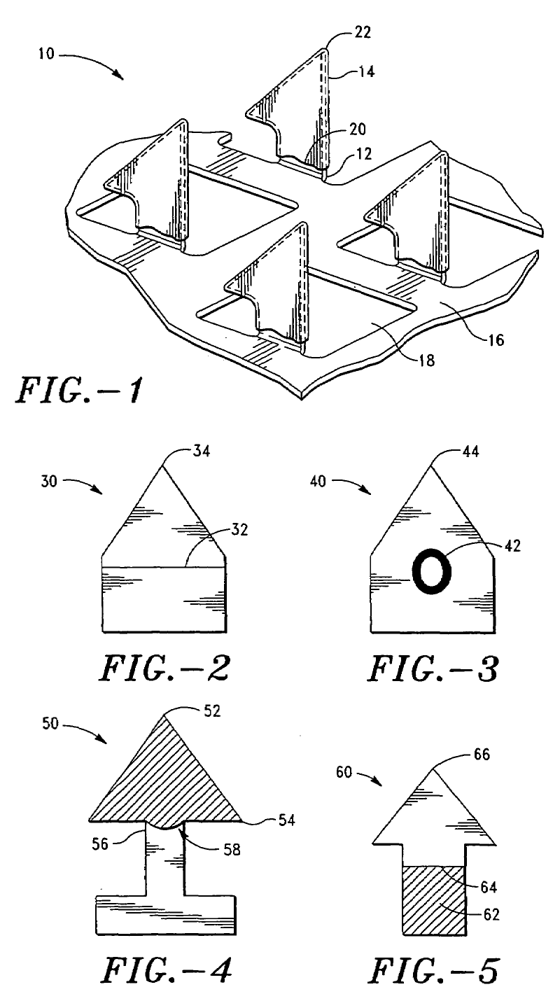

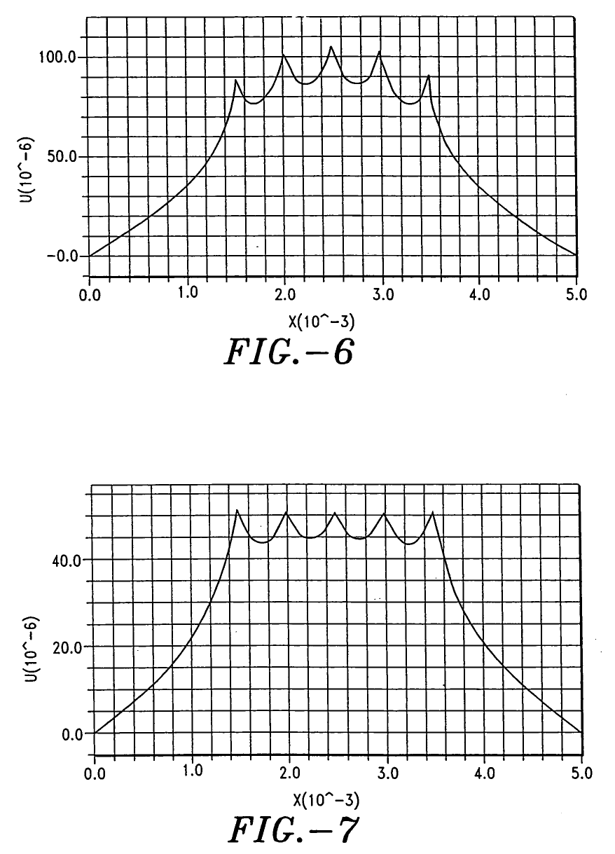

In another embodiment of the invention, the microprojection is configured to minimize the effect of capillary action to wick fluid beyond a desired region. As shown in FIG. 4, microprojection 50 has a width that increases from distal tip 52 to location 54 of maximum width. The width of microprojection 50 then decreases to location 56 of minimum width. The capillary control feature is the reduction of width at location 56. Preferably, location 56 corresponds to the desired coating depth. As shown in FIG. 4, coating 58 wicking action causes migration only in the minimum width 56 location. Microprojection 50 still presents adequate surface area below minimum width 56 to allow a desired amount of coating. As one having skill in the art can appreciate, decreasing the width of the microprojection reduces variability in coating height but must be balanced against the need to retain sufficient structural integrity.

Preferably, the maximum width at location 54 is in the range of approximately 10 μm to 120 μm wider than the minimum width at location 56. Alternatively, the minimum width at location 56 of the microprojection is preferably in the range of approximately 25% to 100%, and more preferably, in the range of approximately 35% to 70%, of the maximum width at location 54. In one presently preferred embodiment, the minimum width at location 56 is approximately 50% of the maximum width at location 54. The reduction to minimum width is located at the desired coating depth, such as in the range of approximately 25 μm to 200 μm from the distal tip of the microprojection.

In another embodiment of the invention, the capillary control feature comprises a hydrophobic coating. As shown in FIG. 5, microprojection 60 has a hydrophobic coating 62 located at the proximal boundary of the location 64 corresponding to the desired coating depth. Preferably, the hydrophobic coating is located in the range of approximately 25 μm to 200 μm from the distal tip 66 of the microprojection. Also preferably, the hydrophobic coating is selected from the group consisting of polytetrafluoroethylene, parylene and silicon.

Presently preferred characteristics of the microprojection members of the invention include a microprojection density in the range of approximately 10 to 2000 per cm², a microprojection length in the range of approximately 50 to 500 μm, a microprojection maximum width in the range of approximately 20 to 300 μm, and a microprojection thickness in the range of approximately 10 to 50 μm.

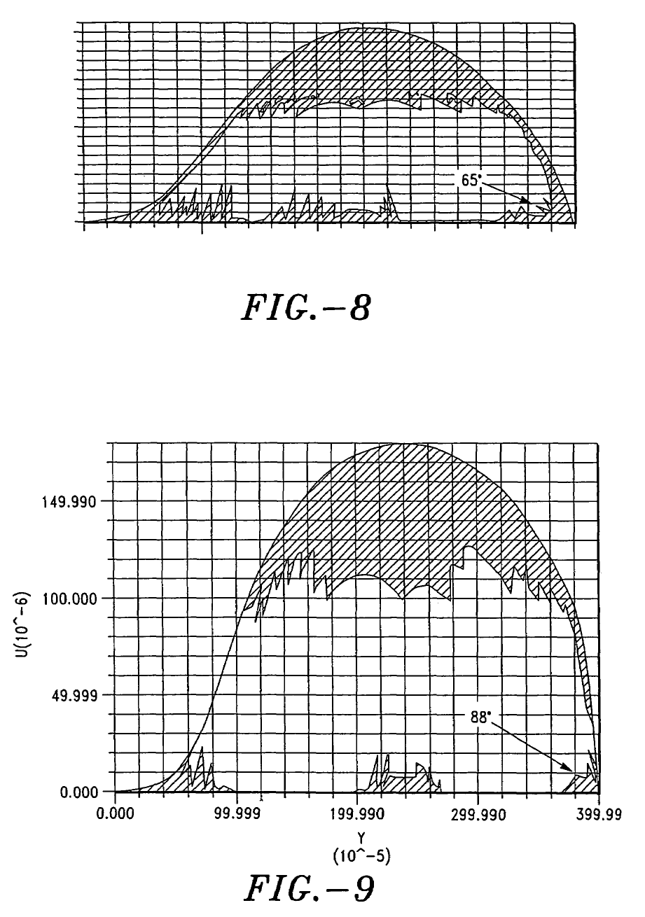

The capillary control features of the invention minimize variations in coating depth as compared to prior art microprojection designs as demonstrated by the graphical illustrations shown in FIGS. 6 and 7. These graphs show the capillary rise measured for five tip microprojection members, each having the same boundary conditions and tip configurations, with FIG. 6 showing prior art designs and FIG. 7 showing microprojections having capillary control features.

As can be seen in FIG. 6, the rise heights show a significant amount of variation, 15 μm or more. The graph also shows that neighboring microprojections affect capillary rise heights, leading to different loading amounts in different positions of the microprojection array.

In contrast, FIG. 7 shows that microprojections having capillary control features offer consistent capillary rise heights and exhibit minimal variability. Also, the position of the microprojection within the array does not have a significant effect on coating depth for designs incorporating capillary control features.

The capillary control features of the invention also significantly increase the potential loading amount. FIGS. 8 and 9 are graphical illustrations that compare the meniscus volume for a conventional microprojection tip with a microprojection tip having capillary control features, respectively, when dipped to a depth of 400 μm. The fluid loading on the tip shown in FIG. 8 is calculated to be 6.3×10⁻¹² m³ as compared to the 36.2×10⁻¹² m³ for the capillary controlled microprojection of FIG. 9. Accordingly, the use of capillary control features can result in approximately a six-fold increase in loading.

Further, without a capillary control feature, the contact angle of the meniscus limits the volume of coating on the microprojection. Microprojections formed from titanium, for example, exhibit a contact angle of approximately 65° as shown in FIG. 8, and microprojections formed from stainless steel have an even lower contact angle. In contrast, the use of a capillary control feature allows the contact angle to approach 90°, effectively removing contact angle as a limiting factor. As shown in FIG. 9, the contact angle with a microprojection having a capillary control feature is approximately 88°. Accordingly, the use of capillary control features allows coatings to be applied to the microprojection at contact angles greater than would be possible without such features.

For example, using the capillary control features of the invention, the coating formulation can be applied with a contact angle greater than approximately 25 degrees. More preferably, the coating formulation can be applied with a contact angle between approximately 30 and 60 degrees.

In one aspect of the invention, the biologically active agent comprises a therapeutic agent in all the major therapeutic areas including, but not limited to, anti-infectives, such as antibiotics and antiviral agents; analgesics, including buprenorphine and analgesic combinations; anesthetics; anorexics; antiarthritics; antiasthmatic agents, such as terbutaline; anticonvulsants; antidepressants; antidiabetic agents; antidiarrheals; antihistamines; anti-inflammatory agents; antimigraine preparations; antimotion sickness preparations, such as scopolamine and ondansetron; antinauseants; antineoplastics; antiparkinsonism drugs; antipruritics; antipsychotics; antipyretics; antispasmodics, including gastrointestinal and urinary; anticholinergics; sympathomimetrics; xanthine derivatives; cardiovascular preparations, including calcium channel blockers such as nifedipine; beta blockers; beta-agonists, such as dobutamine and ritodrine; antiarrythmics; antihypertensives, such as atenolol; ACE inhibitors, such as ranitidine; diuretics; vasodilators, including general, coronary; peripheral, and cerebral; central nervous system stimulants; cough and cold preparations; decongestants; diagnostics; hormones, such as parathyroid hormone; hypnotics; immunosuppressants; muscle relaxants; parasympatholytics; parasympathomimetrics; prostaglandins; proteins; peptides; psychostimulants; sedatives; and tranquilizers. Other suitable agents include vasoconstrictors, anti-healing agents and pathway patency modulators. One or more biologically active agents can also be combined as desired.

In a preferred embodiment, the biologically active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8]arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

Other suitable biologically active agents comprise immunologically active agents, such as vaccines and antigens in the form of proteins, polysaccharide conjugates, oligosaccharides, and lipoproteins. Specific subunit vaccines in include, without limitation, Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from BPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), and Vibrio cholerae (conjugate lipopolysaccharide).

Suitable immunologically active agents also include nucleic acids, such as single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

For storage and application (in accordance with one embodiment of the invention), the microprojection member 10 is preferably suspended in a retainer ring by adhesive tabs, as described in detail in Co-Pending U.S. application Ser. No. 09/976,762 (Pub. No. 2002/0091357), which is incorporated by reference herein in its entirety.

After placement of the microprojection member 10 in the retainer ring, the microprojection member 10 is applied to the patient's skin. Preferably, the microprojection member 10 is applied to the skin using an impact applicator, such as disclosed in Co-Pending U.S. application Ser. No. 09/976,798, which is incorporated by reference herein in its entirety.

From the foregoing description, one of ordinary skill in the art can easily ascertain that the present invention, among other things, provides an effective and efficient means for enhancing the transdermal flux of a biologically active agent into and through the stratum corneum of a patient.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A transdermal delivery device comprising a microprojection member having at least one stratum corneum-piercing microprojection having a capillary control feature, wherein said microprojection has a length running from a distal tip to a proximal end and a thickness, wherein said capillary control feature is located between said distal tip and said proximal end, and wherein said microprojection has a first width at said capillary control feature location.

2. The device of claim 1, wherein said capillary control feature is located in the range of approximately 25 μm to 200 μm from said distal tip of said microprojection.

3. The device of claim 1, wherein said capillary control feature comprises a scribe line running perpendicular to said microprojection length.

4. The device of claim 3, wherein said scribe line extends at least 50% of said first width on each side of said microprojection.

5. The device of claim 3, wherein said scribe line comprises a ridge.

6. The device of claim 3, wherein said scribe line comprises a trough.

7. The device of claim 3, wherein said scribe line has a thickness in the range of approximately 5 μm and 25% of said thickness of said microprojection.

8. The device of claim 1, wherein said capillary control feature comprises at least one void.

9. The device of claim 8, wherein said void has a horizontal dimension up to approximately half said first width.

10. The device of claim 1, wherein said capillary control feature comprises a transition from a maximum width to said first width at said capillary control feature location.

11. The device of claim 10, wherein said maximum width is in the range of approximately 10 μm to 120 μm wider than said first width.

12. The device of claim 10, wherein said first width is in the range of approximately 25% to 100% of said maximum width.

13. The device of claim 12, wherein said first width is in the range of approximately 35% to 70% of said maximum width.

14. The device of claim 13, wherein said first width is approximately 50% of said maximum width.

15. The device of claim 10, wherein said first width is in the range of approximately 10 μm to 120 μm less than said maximum width.

16. The device of claim 1, wherein said capillary control feature comprises a hydrophobic coating.

17. The device of claim 1, further comprising a coating of a biologically active agent applied to said microprojection from said distal tip to said capillary control feature.

18. The device of claim 17, wherein said coating is applied to said formulation with a contact angle at said capillary control feature greater than approximately 25 degrees.

19. The device of claim 18, wherein said coating is applied to said formulation with a contact angle at said capillary control feature approximately between 30 and 60 degrees.

20. The device of claim 17, wherein said biologically active agent is selected from the group consisting of growth hormone release hormone (GHRH), growth hormone release factor (GHRF), insulin, insultropin, calcitonin, octreotide, endorphin, TRN, NT-36 (chemical name: N-[[(s)-4-oxo-2-azetidinyl]carbonyl]-L-histidyl-L-prolinamide), liprecin, pituitary hormones (e.g., HGH, HMG, desmopressin acetate, etc), follicle luteoids, aANF, growth factors such as growth factor releasing factor (GFRF), bMSH, GH, somatostatin, bradykinin, somatotropin, platelet-derived growth factor releasing factor, asparaginase, bleomycin sulfate, chymopapain, cholecystokinin, chorionic gonadotropin, erythropoietin, epoprostenol (platelet aggregation inhibitor), gluagon, HCG, hirulog, hyaluronidase, interferon alpha, interferon beta, interferon gamma, interleukins, interleukin-10 (IL-10), erythropoietin (EPO), granulocyte macrophage colony stimulating factor (GM-CSF), granulocyte colony stimulating factor (G-CSF), glucagon, leutinizing hormone releasing hormone (LHRH), LHRH analogs (such as goserelin, leuprolide, buserelin, triptorelin, gonadorelin, and napfarelin, menotropins (urofollitropin (FSH) and LH)), oxytocin, streptokinase, tissue plasminogen activator, urokinase, vasopressin, deamino [Val4, D-Arg8] arginine vasopressin, desmopressin, corticotropin (ACTH), ACTH analogs such as ACTH (1-24), ANP, ANP clearance inhibitors, angiotensin II antagonists, antidiuretic hormone agonists, bradykinn antagonists, ceredase, CSI's, calcitonin gene related peptide (CGRP), enkephalins, FAB fragments, IgE peptide suppressors, IGF-1, neurotrophic factors, colony stimulating factors, parathyroid hormone and agonists, parathyroid hormone antagonists, parathyroid hormone (PTH), PTH analogs such as PTH (1-34), prostaglandin antagonists, pentigetide, protein C, protein S, renin inhibitors, thymosin alpha-1, thrombolytics, TNF, vasopressin antagonists analogs, alpha-1 antitrypsin (recombinant), and TGF-beta.

21. The device of claim 17, wherein said biologically active agent comprises an immunologically active agent selected from the group consisting of proteins, polysaccharide conjugates, oligosaccharides, lipoproteins, subunit vaccines, Bordetella pertussis (recombinant PT accince—acellular), Clostridium tetani (purified, recombinant), Corynebacterium diphtheriae (purified, recombinant), Cytomegalovirus (glycoprotein subunit), Group A streptococcus (glycoprotein subunit, glycoconjugate Group A polysaccharide with tetanus toxoid, M protein/peptides linked to toxing subunit carriers, M protein, multivalent type-specific epitopes, cysteine protease, C5a peptidase), Hepatitis B virus (recombinant Pre S1, Pre-S2, S, recombinant core protein), Hepatitis C virus (recombinant—expressed surface proteins and epitopes), Human papillomavirus (Capsid protein, TA-GN recombinant protein L2 and E7 [from HPV-6], MEDI-501 recombinant VLP L1 from HPV-11, Quadrivalent recombinant BLP L1 [from HPV-6], HPV-11, HPV-16, and HPV-18, LAMP-E7 [from HPV-16]), Legionella pneumophila (purified bacterial survace protein), Neisseria meningitides (glycoconjugate with tetanus toxoid), Pseudomonas aeruginosa (synthetic peptides), Rubella virus (synthetic peptide), Streptococcus pneumoniae (glycoconjugate [1, 4, 5, 6B, 9N, 14, 18C, 19V, 23F] conjugated to meningococcal B OMP, glycoconjugate [4, 6B, 9V, 14, 18C, 19F, 23F] conjugated to CRM197, glycoconjugate [1, 4, 5, 6B, 9V, 14, 18C, 19F, 23F] conjugated to CRM1970, Treponema pallidum (surface lipoproteins), Varicella zoster virus (subunit, glycoproteins), Vibrio cholerae (conjugate lipopolysaccharide), whole virus, bacteria, weakened or killed viruses, cytomegalo virus, hepatitis B virus, hepatitis C virus, human papillomavirus, rubella virus, varicella zoster, weakened or killed bacteria, bordetella pertussis, clostridium tetani, corynebacterium diphtheriae, group A streptococcus, legionella pneumophila, neisseria meningitidis, pseudomonas aeruginosa, streptococcus pneumoniae, treponema pallidum, vibrio cholerae, flu vaccines, Lyme disease vaccine, rabies vaccine, measles vaccine, mumps vaccine, chicken pox vaccine, small pox vaccine, hepatitis vaccine, pertussis vaccine, diphtheria vaccine, nucleic acids, single-stranded and double-stranded nucleic acids, supercoiled plasmid DNA, linear plasmid DNA, cosmids, bacterial artificial chromosomes (BACs), yeast artificial chromosomes (YACs), mammalian artificial chromosomes, and RNA molecules.

22. A method for applying a coating of a biologically active agent to a transdermal delivery device comprising the steps of providing a microprojection member having at least one stratum corneum-piercing microprojection having a capillary control feature, wherein said microprojection has a length running from a distal tip to a proximal end, a thickness, wherein said capillary control feature is located between said distal tip and said proximal end, and wherein said microprojection has a first width at said capillary control feature location; applying a formulation of said biologically active agent to a location proximal said distal tip of said microprojection so that said formulation migrates to said capillary control feature; and drying said formulation to form a coating.

23. The method of claim 20, wherein the step of applying said formulation comprises dip coating.

24. The method of claim 22, wherein the step of applying a formulation comprises applying a formulation with a contact angle at said capillary control feature greater than approximately 25 degrees.

25. The method of claim 24, wherein the step of applying a formulation comprises applying a formulation with a contact angle at said capillary control feature approximately between 30 and 60 degrees.
